(19) United States
(12) Patent Application Publication
(10) Pub. No.: US 2026/0207722 A1
(43) Pub. Date: Jul. 23, 2026

(54) COMBINATION FOR TREATMENT OF AUTOIMMUNE DISEASES

(71) Applicant: Bonsai Biotherapeutics AB, Göteborg (SE)

(72) Inventors: Charlotte FRIBERT, Göteborg (SE); Björn LÖWENADLER, Askim (SE); Maria FERNANDEZ, Lindome (SE)

(21) Appl. No.: 19/163,158

(22) PCT Filed: Mar. 6, 2024
(86) PCT No.: PCT/SE2024/050208

§ 371 Date: Sep. 8, 2025

(30) Foreign Application Priority Data
Mar. 9, 2023 (SE) 2350260-2

(51) Int. Cl. A61K 39/00; A61K 31/196; A61K 31/573; A61P 21/04; C07K 7/08; C07K 14/705

(52) U.S. Cl. CPC A61K 39/0008; A61K 31/196; A61K 31/573; A61P 21/04; C07K 7/08; C07K 14/70571; C07K 2319/00

(57) ABSTRACT

The invention relates to a combination comprising an effective amount of a tolerogen and an effective amount of an immunomodulator, wherein the tolerogen preferably induces immunological tolerance to skeletal muscle acetylcholine receptor subunit alpha and the immunomodulator preferably is an immunosuppressant, preferably methylprednisone, or a non-selective COX-inhibitor, preferably diclofenac. The invention also relates to the use of such combinations in prevention or treatment of an autoimmune disease, particularly myasthenia gravis.

### TECHNICAL FIELD

The present invention generally relates to a combination of a tolerogen and an immunomodulator, and in particular to the use thereof in the treatment of autoimmune diseases.

### BACKGROUND

An autoimmune disease is a condition arising from an abnormal immune response to a functioning body part. The human immune system typically produces both T cells and B cells that are capable of being reactive with self-entities, but these self-reactive cells are usually either killed prior to becoming active within the immune system, placed into a state of anergy (silently removed from their role within the immune system due to over-activation), or removed from their role within the immune system by regulatory cells. When any one of these control mechanisms fail, an autoimmune disease may develop wherein an affected subject's self-reactive immune cells become active and attack healthy cells in various organs and tissues. There are over 100 identified autoimmune disorders, most of which are chronic conditions, with vastly varying symptoms depending on the specific body constituent that is being attacked.

As an example, myasthenia gravis is a relatively rare autoimmune nerve-muscle disease that causes severe muscle weakness and, in many cases, a difficult life situation for the affected subject. Characteristics of the disease are fatigue and muscle weakness caused by an impaired transmission of nerve impulses to muscles. This is due to an immune attack directed against the skeletal muscle acetylcholine receptor (AChR), which is the relay station of muscles and the receiver of signals from the nerve. Destruction of AChR leads to defective neuromuscular transmission and a subsequent muscle weakness, which can be very serious for the subject.

There are currently no treatments available that target the root cause of the disease. Rather, today, patients with myasthenia gravis are treated with symptom-relieving therapies, such as acetylcholinesterase inhibitors or immunosuppressive therapy, and, in many cases, thymectomy—a complicated surgical procedure, which removes the thymus. Common denominators of current treatments are that they are all symptomatic, have a limited effectiveness and can cause serious side effects.

Therefore, there is a large unmet need for new and effective treatment options to offer subjects suffering from autoimmune diseases, such as myasthenia gravis.

WO 2022/260579 discloses a method of producing a fusion protein between an extracellular domain of the skeletal muscle acetylcholine receptor subunit alpha 1 (nAChRα1) and a solubility enhancing peptide. The method comprises solubilizing inclusion bodies comprising the fusion protein in a solubilization solution having a pH of at least 11 to form solubilized fusion proteins. The method also comprises diluting the solubilized fusion protein in a refolding solution having a pH of no more than 9 to form a refolded monomeric form of the fusion protein.

### SUMMARY

It is a general objective to provide safe and effective treatments for autoimmune diseases, such as myasthenia gravis.

This and other objectives are met by embodiments of the present invention.

An aspect of the invention relates to a combination comprising an effective amount of a tolerogen and an effective amount of an immunomodulator.

Further aspects of the invention relate to a combination according to above for use as a medicament or for use in prevention or treatment of an autoimmune disease. The tolerogen induces, when administered to a subject suffering from the autoimmune disease or having a risk of developing the autoimmune disease, immunological tolerance to a self-entity of the subject that the subject's immune system incorrectly attacks in the autoimmune disease.

The combination of a tolerogen and an immunomodulator achieved a significantly improved effect in treatment of autoimmune diseases, such as myasthenia gravis, as compared to administering only the tolerogen or only the immunomodulator. The two active agents of the combination thereby synergistically improve treatment of autoimmune diseases.

### BRIEF DESCRIPTION OF THE DRAWINGS

The embodiments, together with further objects and advantages thereof, may best be understood by making reference to the following description taken together with the accompanying drawings, in which:

FIG. 1A is a diagram illustrating experimental autoimmune myasthenia gravis (EAGM) scores in rats treated with TOL2, methylprednisolone, or TOL2 and methylprednisolone at 7 days following induction of EAGM.

FIG. 1B is a diagram illustrating weight change of rats treated with TOL2, methylprednisolone, or TOL2 and methylprednisolone at 7 days following induction of EAGM.

FIG. 2A is a diagram illustrating EAGM scores in rats treated with TOL2, methylprednisolone, or TOL2 and methylprednisolone at 40 days following induction of EAGM.

FIG. 2B is a diagram illustrating weight change of rats treated with TOL2, methylprednisolone, or TOL2 and methylprednisolone at 40 days following induction of EAGM.

FIG. 3A is a diagram illustrating EAGM scores in rats treated with TOL2, diclofenac, or TOL2 and diclofenac at 40 days following induction of EAGM.

FIG. 3B is a diagram illustrating weight change of rats treated with TOL2, diclofenac, or TOL2 and diclofenac at 40 days following induction of EAGM.

FIG. 4A is a diagram illustrating serum anti-AChR antibody titer in rats treated with TOL2, methylprednisolone, or TOL2 and methylprednisolone at 7 days following induction of EAGM.

FIG. 4B is a diagram illustrating serum anti-AChR antibody titer in rats treated with TOL2, methylprednisolone, or TOL2 and methylprednisolone at 40 days following induction of EAGM.

### DETAILED DESCRIPTION

The present invention generally relates to a combination of a tolerogen and an immunomodulator, and in particular to the use thereof in the treatment of an autoimmune disease.

The present invention is based on the surprising effects achieved by combining tolerogen treatment with immunomodulator treatment in subjects suffering from an autoimmune disease. Such a combination of a tolerogen and an immunomodulator led to a significantly improved effect in an in vivo autoimmune disease model as compared treatment with only the tolerogen or only the immunomodulator. In fact, treatment with the immunomodulator alone generally did not show any improvement at all in the in vivo autoimmune disease model. However, when combined with the tolerogen, the immunomodulator boosted the positive effects of the tolerogen so that the combination led to a significant improvement over only tolerogen treatment.

These improved effects of the combination was surprising since the immunomodulator as such did generally not induce any improvement in the in vivo autoimmune disease model. A possible explanation for the synergistic effects achieved by the combination is that the tolerance restoring effects of the tolerogen are enhanced in a functional or normalized immune system of a subject, i.e., when the activity of the immune system in an autoimmune disease is suppressed. This is seen in the EXAMPLE section where the effects of the combination of the tolerogen and an immunomodulator are in particular enhanced when administered to subjects suffering from the autoimmune disease, i.e., treatment, as compared to when administered to subjects prior to development of clinical signs of the autoimmune disease, i.e., prophylaxis. Thus, the combination is particularly effective when used for treatment of an autoimmune disease as compared to prevention of an autoimmune disease. It is thus believed that the immunomodulator curbes the activated immune system of subjects suffering from autoimmune diseases to enable the tolerogen to induce tolerance against the self-entity, against which an abnormal immune response is directed causing the autoimmune disease.

An aspect of the invention relates to a combination comprising an effective amount of a tolerogen and an effective amount of an immunomodulator.

A tolerogen is an entity, such as e.g., a protein or peptide, that restores tolerance to a body's own constituents (self-constituents or self-entities) that the immune system incorrectly attacks in an autoimmune disease. This means that the tolerogen invokes a specific immune non-responsiveness against the particular self-constituent or self-entity, against which an abnormal immune response is raised causing the autoimmune disease. Tolerogens differ from immunogens, which are entities, such as foreign proteins, that induce immunity against themselves. In an example, the self-entity could be a protein or a complex comprising at least one protein or peptide molecule. In such a case, the self-entity could be defined as a self-protein. Also, other types of self-entities than proteins and peptides could be targeted by the immune system in an autoimmune disease. Such other types of self-entities include nucleic acid molecules, polysaccharides, etc.

A tolerogen thereby induces, when administered to a subject suffering from an autoimmune disease or having a risk of developing an autoimmune disease, immunological tolerance to a self-entity (frequently a self-protein) of the subject that the subject's immune system incorrectly attacks in the autoimmune disease.

A self-protein, or endogenous protein, as used herein refers to a protein endogenously produced by transcription and translation within the subject of a gene encoding the self-protein and present in the genome of the subject. Non-self or foreign proteins are proteins that are not encoded by the genome of the subject but can enter the subject's body or be expressed therein by pathogens or other organisms present in the body of the subject. These non-self or foreign proteins may be targeted and attacked by the immune system of the subject. The immune system of a healthy subject should not target self-entities, such as self-proteins, as tolerance to such self-entities is crucial for maintaining homeostasis. In an autoimmune disease, the immune system erroneously identifies a self-entity as non-self and an abnormal immune response is raised against the self-entity.

A tolerogen preferably comprises at least a portion of the self-entity, against which an abnormal immune response is raised. Thus, in case the self-entity is a self-protein, a tolerogen is preferably in the form of a polypeptide comprising at least a portion of a self-protein. The tolerogen may comprise a polypeptide of consecutive amino acid residues corresponding to a sub-portion of the self-protein, or multiple, i.e., at least two, copies of such a polypeptide. A tolerogen could also be in the form of a fusion protein of different polypeptides or portions of the self-protein fused together and where these polypeptides or portions are not adjacent to each other in the native self-protein. It is also possible to have a tolerogen in the form of a fusion protein of at least one polypeptide or portion of the self-protein and at least one polypeptide of another polypeptide from another self-protein, a foreign protein or indeed a synthetic polypeptide.

A tolerogen is typically specific for a given autoimmune disease. Thus, a first tolerogen is used in the combination for treatment of a first autoimmune disease, whereas a second, different tolerogen is used in the combination for treatment of a second, different autoimmune disease, and so on.

In an embodiment, the autoimmune disease is myasthenia gravis and the tolerogen induces immunological tolerance to the skeletal muscle acetyl choline receptor subunit alpha 1 (nAChRα1).

AChR is an integral multi-subunit, membrane-spanning receptor comprising an ion channel that responds to the binding of the neurotransmitter acetylcholine. AChRs are typically classified as nicotinic acetylcholine receptors (nAChR) that are particularly responsive to nicotine and muscarinic acetylcholine receptors (mAChR) that are particularly responsive to muscarine. nAChRs are found in the central and peripheral nervous system, muscle, and many other tissues in the human body. At the neuromuscular junction they are the primary receptor in muscle for the nerve-muscle communication that controls muscle contraction.

nAChR is made up of five subunits arranged symmetrically around a central pore. Each subunit comprises four transmembrane domains with both the N-terminal and the C-terminal located extracellularly. In humans and other vertebrates, nAChR are broadly classified into two subtypes; muscle-type nAChR and neuronal-type nAChR. Muscle-type nAChRs found at the neuromuscular junctions are either in an embryonic form, composed of α₁, β₁, γ, and δ subunits in a 2:1:1:1 ratio ((α₁)₂β₁γδ), or the adult form composed of α₁, β₁, δ, and ε subunits in a 2:1:1:1 ratio ((α₁)₂β₁δε).

In humans, muscle-type nAChRα1 is encoded by the CHRNA1 gene and is presented below and in SEQ ID NO: 1:

MEPWPLLLLF SLCSAGLVLG SEHETRLVAK LFKDYSSVVR
PVEDHRQVVE VTVGLQLIQL INVDEVNQIV TINVRLKQQW
VDYNLKWNPD DYGGVKKIHI PSEKIWRPDL VLYNNADGDF
AIVKFTKVLL QYTGHITWTP PAIFKSYCEI IVTHFPFDEQ
NCSMKLGTWT YDGSVVAINP ESDQPDLSNF MESGEWVIKE
SRGWKHSVTY SCCPDTPYLD ITYHFVMQRL PLYFIVNVII
PCLLESFLTG LVFYLPTDSG EKMTLSISVL LSLTVELLVI
VELIPSTSSA VPLIGKYMLE TMVFVIASII ITVIVINTHH
RSPSTHVMPN WVRKVFIDTI PNIMFFSTMK RPSREKQDKK
IFTEDIDISD ISGKPGPPPM GFHSPLIKHP EVKSAIEGIK
YIAETMKSDQ ESNNAAAEWK YVAMVMDHIL LGVEMLVCII
GTLAVFAGRL IELNQQG

An isoform (splice variant) of the muscle-type nAChRα1 comprises 25 extra amino acids marked in underlining below (SEQ ID NO: 9):

MEPWPLLLLF SLCSAGLVLG SEHETRLVAK LFKDYSSVVR
PVEDHRQVVE VTVGLQLIQL INVDEVNQIV TTNVRLKQGD
MVDLPRPSCV TLGVPLFSHL QNEQWVDYNL KWNPDDYGGV
KKIHIPSEKI WRPDLVLYNN ADGDFAIVKF TKVLLQYTGH
ITWTPPAIFK SYCEIIVTHF PFDEQNCSMK LGTWTYDGSV
VAINPESDQP DLSNEMESGE WVIKESRGWK HSVTYSCCPD
TPYLDITYHF VMQRLPLYFI VNVIIPCLLF SFLTGLVFYL
PTDSGEKMTL SISVLLSLTV FLLVIVELIP STSSAVPLIG
KYMLFTMVFV IASIIITVIV INTHHRSPST HVMPNWVRKV
FIDTIPNIMF FSTMKRPSRE KQDKKIFTED IDISDISGKP
GPPPMGFHSP LIKHPEVKSA IEGIKYIAET MKSDQESNNA
AAEWKYVAMV MDHILLGVEM LVCIIGTLAV FAGRLIELNQ
QG

In an embodiment, the tolerogen is a fusion protein comprising an extracellular domain of nAChRα1 and a solubility enhancing peptide.

The extracellular domain of nAChRα1 corresponds to amino acids 21 to 230 of SEQ ID NO: 1 and is presented below and in SEQ ID NO: 3:

SEHETRLVAK LFKDYSSVVR PVEDHRQVVE VTVGLQLIQL
INVDEVNQIV TTNVRLKQQW VDYNLKWNPD DYGGVKKIHI
PSEKIWRPDL VLYNNADGDE AIVKFTKVLL QYTGHITWTP
PAIFKSYCEI IVTHFPFDEQ NCSMKLGTWT YDGSVVAINP
ESDQPDLSNF MESGEWVIKE SRGWKHSVTY SCCPDTPYLD
ITYHFVMQRL

The extracellular domain of nAChRα1 also corresponds to amino acids 21 to 255 of SEQ ID NO: 9 and is presented below and in SEQ ID NO: 2:

SEHETRLVAK LFKDYSSVVR PVEDHRQVVE VTVGLQLIQL
INVDEVNQIV TTNVRLKQGD MVDLPRPSCV TLGVPLFSHL
QNEQWVDYNL KWNPDDYGGV KKIHIPSEKI WRPDLVLYNN
ADGDFAIVKF TKVLLQYTGH ITWTPPAIFK SYCEIIVTHF
PFDEQNCSMK LGTWTYDGSV VAINPESDQP DLSNEMESGE
WVIKESRGWK HSVTYSCCPD TPYLDITYHF VMQRL

In an embodiment, the tolerogen is a fusion protein based on SEQ ID NO: 3 (amino acid residues 1-58, 84-235 in SEQ ID NO: 2) but in which twelve amino acids (marked in bold above) of a sequence motif denoted the “Cys loop” have been exchanged to improve solubility properties of the fusion protein and promote effective recombinant expression while retaining the native structure.

In an embodiment, the twelve amino acids at position 129 to 140 in SEQ ID NO: 3 that is part of the sequence motif denoted the “Cys-loop” between Cys128 and Cys142 have been exchanged by the solubility enhancing peptide. In a particular embodiment, these twelve amino acids at position 129 to 140 in SEQ ID NO: 3 have been replaced by twelve amino acids Asp132-Thr143 (DVSGVDTESGAT, SEQ ID NO: 4) from the homologous acetylcholine binding-protein (AChBP) of Lymnaea stagnalis. This amino acid sequence exchange has been made in order to improve solubility properties and promote effective recombinant expression while retaining the native structure. The fusion protein comprising the amino acid sequence Glu129-Gln140 in SEQ ID NO: 3 replaced by the amino acid sequence Asp132-Thr143 from L. stagnalis AChBP is presented below and in SEQ ID NO: 5:

SEHETRLVAK LFKDYSSVVR PVEDHRQVVE VTVGLQLIQL
INVDEVNQIV TTNVRLKQQW VDYNLKWNPD DYGGVKKIHI
PSEKIWRPDL VLYNNADGDF AIVKFTKVLL QYTGHITWTP
PAIFKSYCDV SGVDTESGAT NCSMKLGTWT YDGSVVAINP
ESDQPDLSNF MESGEWVIKE SRGWKHSVTY SCCPDTPYLD
ITYHFVMQRL

In an embodiment, a single N-terminal methionine (M) is added to the amino acid sequence in SEQ ID NO: 5 to enable bacterial expression of the fusion protein, such as in Escherichia coli. The resulting 211 amino acid fusion protein is presented below and in SEQ ID NO: 6:

MSEHETRLVA KLFKDYSSVV RPVEDHRQVV EVTVGLQLIQ
LINVDEVNQI VTTNVRLKQQ WVDYNLKWNP DDYGGVKKIH
IPSEKIWRPD LVLYNNADGD FAIVKFTKVL LQYTGHITWT
PPAIFKSYCD VSGVDTESGA TNCSMKLGTW TYDGSVVAIN
PESDQPDLSN FMESGEWVIK ESRGWKHSVT YSCCPDTPYL
DITYHFVMQR L

The fusion protein has four Cys residues forming two intramolecular disulfide bonds between Cys128-Cys142 and Cys192-Cys193, respectively with the amino acid numbering in accordance with SEQ ID NO: 3 and 5. The fusion protein has a molecular weight of 24194.47 Da, i.e., about 24 kDa, and a theoretical isoelectric pH of 5.33.

In an embodiment, the asparagine residue 141 (Asp141) in SEQ ID NO: 3 (and 5) is not glycosylated. Asp141 forms, with the following two amino acids Cys142 and Ser142, a N-X-S sequon, which otherwise may be involved in N-linked glycosylation. However, in a preferred embodiment, no such N-linked glycosylation occurs on Asp141.

Recombinant production of the fusion protein is complicated by the formation of dimeric and higher multimeric forms of the fusion protein, including aggregates of the fusion proteins. Such higher multimeric forms are generally not desired when using the fusion protein in treatment of myasthenia gravis. Hence, in an embodiment, the fusion protein is in monomeric form or dimeric form, preferably in monomeric form.

The fusion protein is effectively expressed and produced in host cells, preferably bacterial cells, such as E. coli cells, and accumulates in high quantities in intracellular inclusion bodies. The inclusion bodies can be solubilized in a solubilization solution having a pH of less than 11 and lacking any reducing agent to form solubilized fusion proteins. The solubilized fusion proteins are then preferably loaded onto an ionexchange resin and eluted form the ion-exchange resin using an elution solution having a pH less than 11 and comprising a reducing agent to form a fusion protein eluate. The pH of the fusion protein eluate is preferably adjusted to at least 11 and the pH-adjusted fusion protein eluate is then diluted in a refolding solution having a pH of no more than 9 to form a refolded monomeric form of the fusion protein.

More information of the production of the fusion protein is presented in WO 2022/260579, the teachings of which with regard to production of the fusion protein is hereby incorporated by reference.

In an embodiment, the tolerogen is selected from the group consisting of SEQ ID NO: 3, 5, 6, 10, and a mixture thereof. SEQ ID NO: 10 presented here below corresponds to SEQ ID NO: 3 but with an N-terminal methionine.

MSEHETRLVA KLEKDYSSVV RPVEDHRQVV EVTVGLQLIQ
LINVDEVNQI VTTNVRLKQQ WVDYNLKWNP DDYGGVKKIH
IPSEKIWRPD LVLYNNADGD FAIVKFTKVL LQYTGHITWT
PPAIFKSYCEI IVTHFPFDE QNCSMKLGTW TYDGSVVAIN
PESDQPDLSN FMESGEWVIK ESRGWKHSVT YSCCPDTPYL
DITYHFVMQR L

In a preferred embodiment, the tolerogen is selected from the group consisting of SEQ ID NO: 5, 6, and a mixture thereof. In another embodiment, the tolerogen is selected from the group consisting of SEQ ID NO: 3, 10, and a mixture thereof.

Immunomodulator as used herein is a molecule that modulates, i.e., regulatory adjusts, the immune system. Examples of such immunomodulation includes activation of the immune system, amplification of the immune system, and attenuation or suppression of the immune system.

In an embodiment, the immunomodulator is an immunosuppressant.

Immunosuppressant, or immunosuppressive substance, as used herein is a molecule that suppresses, inhibits or prevents activity of the immune system.

In an embodiment, the immunosuppressant is an anti-inflammatory agent. Anti-inflammatory agents are substances that reduce inflammation. In such an embodiment, the immunosuppressant has both immunosuppressive effects and anti-inflammatory effects. A preferred example of such substances are corticosteroids, and in particular glucocorticoids.

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiological processes, including stress response, immune response, regulation of inflammation, carbohydrate metabolism, protein catabolism blood electrolyte levels, and behavior.

Glucocorticoids have, among their immunomodulatory activities, anti-inflammatory and immunosuppressive effects. Anti-inflammatory effects are mediated by blocking the action of inflammatory mediators (transrepression) and inducing anti-inflammatory mediators (transactivation). Anti-inflammatory effects of glucocorticoids is at least partly mediated by regulatory T (Treg) cells. Immunosuppressive effects are mediated at least partly by suppressing the activity of T-lymphocytes. Glucocorticoids affect cells by binding to the glucocorticoid receptor. The activated glucocorticoid receptor-glucocorticoid complex up-regulates the expression of genes encoding anti-inflammatory proteins in the nucleus (transactivation) and represses the expression genes encoding proinflammatory proteins in the cytosol by preventing the translocation of other transcription factors from the cytosol into the nucleus (transrepression).

There are various glucocorticoids that could be used in the combination of the invention including, but not limited to, cortisol (hydrocortisone), cortisone, prednisone, prednisolone, methylprednisolone, dexamethasone, betamethasone, triamcinolone, deflazacort, fludrocortisone acetate, deoxycorticosterone acetate, aldosterone and beclomethasone. A currently preferred glucocorticoid is methylprednisolone.

In an embodiment, the anti-inflammatory agent is a nonsteroidal anti-inflammatory agent (NSAID). NSAIDs work by inhibiting the activity of cyclooxygenase (COX) enzymes (COX-1 and COX-2). In cells, these enzymes are involved in the synthesis of prostaglandins, which are involved in inflammation, and thromboxanes, which are involved in blood clotting. There are two general types of NSAIDs: non-selective COX inhibitors, and selective COX inhibitors. Non-selective COX inhibitors inhibit the activity of both COX-1 and COX-2. Selective COX inhibitors are COX-2 selective inhibitors.

In an embodiment, the NSAID is selected from the group consisting of a nonselective COX inhibitor and a selective COX inhibitor (COX-2 selective inhibitor).

Illustrative, but non-limiting, examples of nonselective COX inhibitors include ibuprofen, naproxen, diclofenac, mefenamic acid and indomethacin. Illustrative, but non-limiting, examples of selective COX inhibitors include celecoxib and etoricoxib.

In a particular embodiment, the NSAID is a nonselective COX inhibitor selected from the group consisting of ibuprofen, naproxen, diclofenac, mefenamic acid and indomethacin. In a preferred embodiment, the nonselective COX inhibitor is diclofenac.

In an embodiment, the immunosuppressant is a T cell activation inhibitor. Such T cell activation inhibitors are capable of down-regulating T cell activity.

In an embodiment, the T cell activation inhibitor is selected from the group consisting of a T-cell immunoglobulin and mucin-domain containing-3 (TIM-3) ligand, a programmed cell death protein 1 (PD-1) ligand, a lymphocyte-activation gene 3 (LAG-3) ligand and a cluster of differentiation 28 (CD28) antagonist.

In an embodiment, the T cell activation inhibitor is a TIM-3 ligand selected from the group consisting of galectin-9, high mobility group protein B1 (HMGB1), carcinoembryonic antigen cell adhesion molecule 1 (CECAM1), and phosphatidylserine.

In an embodiment, the T cell activation inhibitor is a PD-1 ligand selected from the group consisting of programmed death 1 ligand 1 (PD1L1), PD1L2, a soluble extracellular domain of PD1L1, and a soluble extracellular domain of PD1L2.

The soluble extracellular domain of PD1L1 preferably corresponds to amino acid residues 19 to 238 of human PD1L1 as shown in SEQ ID NO: 7 and further here below:

FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER

The soluble extracellular domain of PD1L2 preferably corresponds to amino acid residues 20 to 220 of human PD1L2 as shown in SEQ ID NO: 8 and further here below:

LFTVTVPKELYIIEHGSNVTLECNFDTGSHVNLGAITASLQKVENDTSPH
RERATLLEEQLPLGKASFHIPQVQVRDEGQYQCIIIYGVAWDYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
T

In an embodiment, the T cell activation inhibitor is a LAG-3 ligand selected from the group consisting of major histocompatibility complex class II (MHC class II), fibrinogen-like protein 1 (FGL-1), alpha-synuclein, galectin-3, and lymph node sinusoidal endothelial cell C-type lectin (LSECtin).

In an embodiment, the T cell activation inhibitor is a CD28 antagonist selected from the group consisting of cytotoxic T-lymphocyte-associated protein 4 (CTLA4), and Abatacept.

In an embodiment, the immunomodulator is a Treg cell activator or inducer. Illustrative, but non-limiting, examples of such Treg cell activators or inducers include interleukin-2 (IL-2), transforming growth factor β (TGF-β), retinoic acid, rapamycin, butyrate and any combinations thereof. In a particular embodiment, the Treg cell activators is rapamycin and/or butyrate.

The combination could comprise an effective amount of one tolerogen and an effective amount of one immunomodulator. In another embodiment, at least two different immunomodulators are used in combination with one tolerogen. In such an embodiment, the at least two different immunomodulators preferably have different mechanisms of action, i.e., achieve immunomodulation when administered to a subject by different mechanisms or modes of action. In further embodiments, the combination comprises at least two different tolerogens and one immunomodulator or at least two different tolerogens and at least two different immunomodulators. In case the self-entity is a self-protein and in the case of at least two different tolerogens, the two or more tolerogens preferably comprise different polypeptides of the same self-protein or different fusion proteins comprising different amino acid sequences from the same self-protein.

In another embodiment, the immunomodulator is a protein or peptide immunomodulator. In such an embodiment, the combination comprises a fusion protein between the tolerogen and the protein or peptide immunomodulator. Such a fusion protein could comprise, from the N-terminal to the C-terminal, the tolerogen followed by the protein or peptide immunomodulator or the protein or peptide immunomodulator followed by the tolerogen. It is also possible to have a peptide linker between the tolerogen and the protein or peptide immunomodulator, such as various GS linkers, or cleavable peptide linkers.

An effective amount, or therapeutically effective amount, of the tolerogen and of the immunomodulator in the combination is an amount that alone, or together with further doses, produces the desired (therapeutic) response. The effective amount to be used will depend, for example, upon the therapeutic objectives, the route of administration, the particular tolerogen and immunomodulator, and the condition of the subject. A suitable dosage of the tolerogen and the immunomodulator for a given subject can be determined by an attending physician, taking into consideration various factors known to modify the action of drugs including severity and type of disease, drug species, body weight, sex, diet, time and route of administration, other medications and other relevant clinical factors. The dosages and schedules may be varied according to the particular disease state and the overall condition of the subject. Suitable doses may also be determined based on the severity of the subject. Suitable doses may also be determined for subgroups of subjects, e.g., based on their heredity and/or pharmocogenetic profile(s).

A combination comprising an effective amount of a tolerogen and an effective amount of an immunomodulator encompasses a dosage form of the tolerogen for use in combination with a distinct dosage form of the immunomodulator. Combination also encompasses a dosage form comprising both the tolerogen and the immunomodulator.

Combined use and combination in the context of the invention therefore also includes a product comprising both the tolerogen and the immunomodulator as discrete separate dosage forms, in separate containers or e.g., in blisters containing both types of drugs in discrete solid dosage units, e.g., in a form in which the dosage units which have to be taken together or which have to be taken within one day are grouped together in a manner which is convenient for the subject. The product itself or as a part of a kit may contain instructions for the simultaneous, sequential or separate administration of the discrete separate dosage units, to a subject. Accordingly, the product may comprise the tolerogen and the immunomodulator as discrete separate dosage forms, in a form which is suitable for sequential, separate and/or simultaneous administration. The compounds, combinations and/or compositions may be provided in a form, which is suitable for sequential (consecutive), separate and/or simultaneous (concurrent) administration to the subject, in any order. For example, the tolerogen may be provided in a form that is suitable for sequential, separate and/or simultaneous administration to the immunomodulator.

Accordingly, the tolerogen may be administered to the subject at the same time or at a different time (before or after) compared to when the immunomodulator is administered. In cases where the tolerogen and the immunomodulator are administered simultaneously, they may be administered as separate compositions that are administered at the same time, or may be administered as a combined composition that includes the tolerogen and the immunomodulator.

Hence, in an embodiment, the tolerogen and the immunomodulator are provided in an administration form for separate administration of the effective amount of the tolerogen and the effective amount of the immunomodulator.

In another embodiment, the tolerogen and the immunomodulator are provided in an administration form for simultaneous administration of the effective amount of the tolerogen and the effective amount of the immunomodulator.

For instance, the combination could comprise a first formulation comprising the effective amount of the tolerogen and a second formulation comprising the effective amount of the immunomodulator. In such a case, the two formulations may be administered separately either simultaneously or sequentially.

The compounds, combinations and/or compositions described herein can be administered to the subject by any conventional route, including oral administration, for example in tablet form, injection or by gradual infusion over time. The administration may, for example, be topical, oral, parenteral, intravenous, intraperitoneal, intramuscular, intravascular, intracavity, intranasal, intracerebral, intratracheal, intralesional, intraperitoneal, rectal, subcutaneous, transdermal, epidural, percutaneous, or by infusion.

For example, suitable forms for oral administration include a tablet or capsule; suitable forms for nasal administration or administration by inhalation include a powder or solution; suitable forms for parenteral injection, including intravenous, subcutaneous, intramuscular, intravascular or infusion, include a sterile solution, suspension or emulsion; suitable forms for topical administration include a patch, an ointment or cream; and suitable forms for rectal administration include a suppository. Alternatively, the route of administration may be by injection.

The compounds, combinations and/or compositions of the present invention are advantageously presented in unit dosage form. Dosage forms (also called unit doses) are pharmaceutical drug products in the form, in which they are marketed for use, with a specific mixture of active ingredients and inactive components (excipients), in a particular configuration, such as a capsule shell, and apportioned into a particular dose. Depending on the route of administration, dosage forms include liquid, solid, and semisolid dosage forms. Common dosage forms include pills, tablets, capsules, drinks or syrups.

The tolerogen and/or the immunomodulator may be part of a composition, such as a pharmaceutical composition, that comprises the compound, i.e., the tolerogen and/or the immunomodulator, and one or more other components. A composition may be a pharmaceutical composition that comprises the tolerogen and/or the immunomodulator and a pharmaceutically acceptable excipient, adjuvant, diluent and/or carrier. Pharmaceutical compositions may routinely contain pharmaceutically acceptable concentrations of salt, buffering agents, preservatives, compatible carriers, supplementary immune potentiating agents, such as adjuvants and cytokines and optionally other therapeutic agents or compounds.

Excipients are natural or synthetic substances formulated alongside an active ingredient, i.e., the tolerogen and/or the immunomodulator, included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, magnesium stearate, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art. Carriers are non-toxic to recipients at the dosages and concentrations employed and are compatible with other ingredients of the formulation. The term carrier denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The present invention also relates to a combination according to the invention for use as a medicament and for use in prevention or treatment of an autoimmune disease. In such a case, the tolerogen induces, when administered to a subject suffering from the autoimmune disease or having a risk of developing the autoimmune disease, immunological tolerance to a self-entity, preferably a self-protein, of the subject that the subject's immune system incorrectly attacks in the autoimmune disease.

In a particular embodiment, the combination is for use in treatment of the autoimmune disease.

In a preferred embodiment, the autoimmune disease is myasthenia gravis.

A related aspect of the invention defines use of a tolerogen and an immunomodulator, such as a combination of a tolerogen and an immunomodulator, for the manufacture of a medicament for prevention or treatment, preferably treatment, of an autoimmune disease. In such a case, the tolerogen induces, when administered to a subject suffering from the autoimmune disease or having a risk of developing the autoimmune disease, immunological tolerance to a self-entity, preferably a self-protein, of the subject that the subject's immune system incorrectly attacks in the autoimmune disease.

In a preferred embodiment, the autoimmune disease is myasthenia gravis.

Treatment or treating as used herein means an approach for obtaining beneficial or desired results, including clinical results. Beneficial or desired clinical results could include, for instance, alleviation or amelioration of one or more symptoms or conditions, diminishment of extent of the autoimmune disease, such as myasthenia gravis, stabilized state of the autoimmune disease, i.e., prevent worsening, preventing spread of the autoimmune disease, delay or slowing of disease progression, amelioration or palliation of the disease state, diminishment of the reoccurrence of the autoimmune disease, and remission. Treatment or treating may also prolong survival as compared to expected survival if not receiving any treatment.

Preventing or prophylaxis as used herein means an approach in which a risk of developing the autoimmune disease is reduced or prevented, including prolonging or delaying the autoimmune disease development. For instance, a patient predisposed to develop a disease, such as due to genetic or hereditary predisposition, could benefit for administration of the polypeptide or a pharmaceutical composition comprising the polypeptide to prevent, reduce the risk of, delaying and/or slowing development of the autoimmune disease.

As mentioned in the foregoing, the combination may be administered to a subject or patient in need thereof in the form of one or pharmaceutical compositions comprising the tolerogen and/or the immunomodulator.

The pharmaceutical composition(s) may additionally comprise one or more pharmaceutically acceptable additives including, but not limited to, carriers, vehicles, diluents, adjuvant, aroma, preservatives and/or excipients. Non-limiting examples of a pharmaceutically acceptable carrier or vehicle is an injection solution, such as saline or a buffered injection solution.

The pharmaceutical composition(s) may, for instance, be in the form of a tablet, a capsule, powder, nanoparticles, a solution, such as an injection solution, a transdermal patch or a suppository.

In an embodiment, the pharmaceutical composition for the tolerogen is an aqueous injection solution comprising the tolerogen at a concentration selected within an interval of from 0.1 up to 10 mg/ml, preferably selected within an interval of from 0.5 up to 7.5 mg/ml and more preferably selected within an interval of from 1 up to 5 mg/mL.

In an embodiment, the aqueous injection solution is selected from the group consisting of saline, phosphate-buffered saline and an aqueous tris(hydroxymethyl)aminomethane (Tris) based injection solution. The aqueous injection solution preferably has a neutral pH or a slight basic pH, such as pH selected within an interval of from 7 up to 9, more preferably selected within an interval of from 7.5 up to 9, and even more preferably selected within an interval of from 8 up to 9.

A currently preferred pharmaceutical composition for the tolerogen is an aqueous injection solution comprising the tolerogen at 3.0 mg/ml in 40 mM Tris, 150 mM NaCl, pH 8.5. A preferred administration mode for the tolerogen is intravenous injection.

The subject or patient is preferably a human subject or patient.

The preferred administration mode of the immunomodulator is dependent on the type of immunomodulator. For instance, diclofenac is preferably administered orally, rectally or by injection, whereas methylprednisolone is preferably administered orally or parenterally.

A further aspect of the invention relates to a method for preventing, inhibiting or treating an autoimmune disease. The method comprises administering an effective amount of a tolerogen and an effective amount of an immunomodulator to a subject in need thereof. In such a case, the tolerogen induces, when administered to a subject suffering from the autoimmune disease or having a risk of developing the autoimmune disease, immunological tolerance to a self-entity of the subject that the subject's immune system incorrectly attacks in the autoimmune disease.

### EXAMPLES

### Example 1

In this example the effects of tolerogen treatment, immunosuppressant treatment, and the combined treatment with a tolerogen and an immunosuppressant in an animal model of an autoimmune disease were investigated.

### Materials and Methods

### Animals

6-week-old female Lewis rats were obtained from Janvier Labs (France). They were maintained in the rodent unit of the Department of Animal Models for Biomedical Research of the Hellenic Pasteur Institute, in plastic cages with wire mesh lids and 4 cm thick wood-shavings bedding (four rats per 1,600 cm² cage). Upon symptom manifestation they were provided with water gels and soft food at the bottom of the cages throughout the remaining duration of the experiment.

### TOL2

TOL2 is a tolerogen useful in treatment of myasthenia gravis. TOL2 is a fusion protein between an extracellular domain of nicotine acetylcholine receptor subunit alpha 1 (nAChRα1) and a solubility enhancing peptide from the homologous acetylcholine binding-protein (AChBP) of Lymnaea stagnalis. TOL2 is typically present as a mixture of the 210 amino acid fusion protein as defined in SEQ ID NO: 5 and the 211 amino acid fusion protein as defined in SEQ ID NO: 6 corresponding to SEQ ID NO: 5 but comprising an N-terminal methionine (M) due to expression in Escherichia coli.

### Synthesis of TOL2

Briefly, Escherichia coli cells were cultured in terrific broth (Thermofisher Scientific, USA) at 37° C. and TOL2 expression was induced with 1 mM isopropyl β-D-1-thiogalactopyranoside (IPTG) at an OD₆₀₀ of about 0.6. Following induction, TOL2 accumulated in inclusion bodies in high quantities. TOL2-containing inclusion bodies were purified by cell-disruption in lysis buffer (0.1 M Tris, 5 mM ethylenediaminetetraacetic acid (EDTA), pH 8.5) followed by repeated washings in 2 M urea, 2% Triton X-100 in lysis buffer and finally solubilized in 40 mM Tris, 8 M urea, 5 mM EDTA, pH 8.5. Refolding of TOL2 was performed in 40 mM Tris, 50 mM NaCl, 1 M urea, 10% glycerol, 5% sucrose pH 8.5 overnight at 4° C. Refolded TOL2 was further purified by anion exchange chromatography on Q Sepharose FF at pH 7.4 and size exclusion chromatography (SEC) on Superdex 200 μg. Following the SEC step, TOL2 purity was >90% with endotoxin levels below <1 EU/mg. The overall yield of purified TOL2 was about 80 mg/L of E. coli culture. Following sterile filtration, TOL2 was frozen in storage buffer (30 mM NaP, 0.3 M NaCl, pH 7.4) and stored at −80° C.

### Induction, Treatment, and Clinical Evaluation of Experimental Autoimmune Myasthenia Gravis (EAMG)

For induction of EAMG, rats were anaesthetized with 2% isoflurane supplemented with oxygen. They were injected subcutaneously in both hind footpads and at three sites in the lower back with a total of 80 mg TOL2, or phosphate buffered saline (PBS) for controls, in complete Freund's adjuvant (CFA) (Becton, Dickinson and Company) supplemented with 2 mg/ml inactivated Mycobacterium tuberculosis H37RA (Becton, Dickinson and Company), in a final volume of 250 ml.

Regarding treatment administration, rats were treated with TOL2 and/or methylprednisolone (Lyo-drol, Vianex) starting 7 or 40 days after EAMG induction. The amount of TOL2 was 100 or 500 μg administered intravenously in a volume of 200 μl in tail vein, and the amount of methylprednisolone was 1 mg injected intraperitoneally in a volume of 100 μl. Control animals received only PBS in all experiments. The rats were monitored once a week for the first four weeks after EAMG induction and daily thereafter.

Body weight was recorded, and clinical score was observed on a flat bench before and after exercise and graded based on the presence of the following symptoms: tremor, hunched posture, reduced strength/mobility and dropped head. Exercise consisted of repetitive grasping and pulling of a 350 g grid while being held by the base of the tail for 30 seconds. EAMG scores were evaluated as follows: 0: normal strength, no symptoms; 1: normal before exercise, symptoms observed after exercise due to fatigue; 2: symptoms present without exercise; 3: severe symptoms at rest, hind limb paralysis, no grip; 4: moribund. To minimize investigator bias, the animals were scored by two investigators, one of which was blinded to the treatment groups, and the average scores were used in the analyses.

### Results

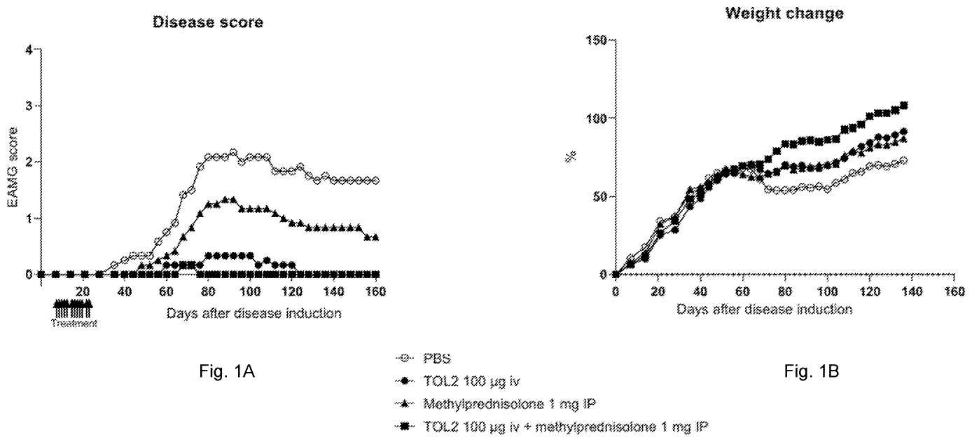

FIG. 1A illustrates the EAMG scores when the treatment started 7 days after EAMG induction. At this early stage, the treatments by TOL2, methylprednisolone and the combination of TOL2 and methylprednisolone represent preventive treatment of EAMG. Intravenous (iv) injection of TOL2 and intraperitoneal (IP) injection of methylprednisolone resulted in a slight improvement in EAMG score as compared to PBS-treated control animals. The improvements were, however, not significant. However, the combined administration of TOL2 and methylprednisolone resulted in a synergistic improvement with EAMG scores corresponding to normal muscle strength and absence of clinical symptoms.

The combination of TOL2 and methylprednisolone also resulted in a larger weight increase in the treated rats during the 100 days trial as compared to PBS control animals or treatment with only TOL2 or only methylprednisolone as shown in FIG. 1B.

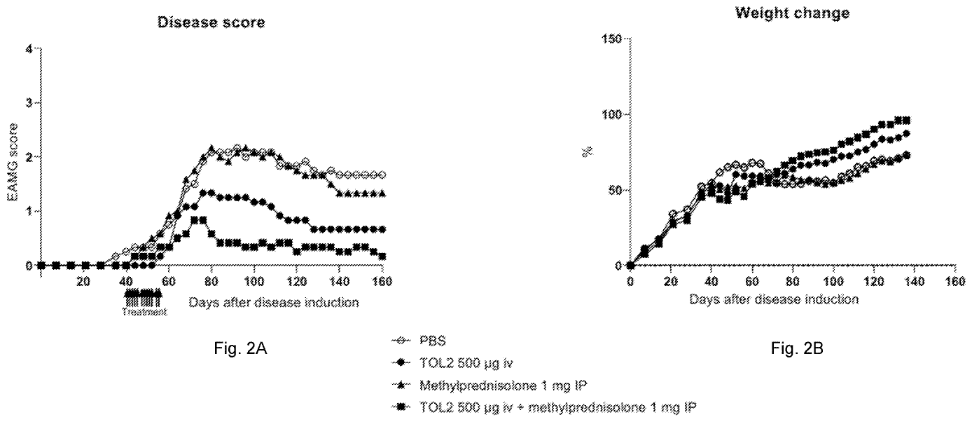

The synergistic effect obtained by the combination of TOL2 and methylprednisolone was even more pronounced when the treatment started 40 days after EAMG induction as shown in FIG. 2A. At this stage, the rats have started to develop symptoms of EAMG and the administration represents a therapeutic treatment of EAMG. Intravenous injection of TOL2 resulted in a slight improvement in EAMG score as compared to PBS-treated control animals. Intraperitoneal injection of methylprednisolone did not show any improvement at all compared in PBS-treated control animals. However, combining intravenous injection of TOL2 and intraperitoneal injection of methylprednisolone resulted in a synergistic improvement of the treated rats. This was highly surprising since methylprednisolone treatment alone did not result in any effects on EAMG scores over PBS-treated control animals. However, when combining TOL2 with methylprednisolone treatment, methylprednisolone synergistically boosted the beneficial effects seen with TOL2 treatment alone. This means that the combination of TOL2 and methylprednisolone achieved a true synergistic effect in the EAMG model.

The combination of TOL2 and methylprednisolone also resulted in a larger weight increase in the rats during the 100 days of the trial compared to PBS-treated control animals or to animals treated with only TOL2 or only methylprednisolone as shown in FIG. 2B.

### Example 2

In this example the effects of tolerogen treatment, immunosuppressant treatment and the combined treatment with a tolerogen and an immunosuppressant in an animal model of an autoimmune disease ere investigated.

### Materials and Methods

### Animals

6- to 7-week-old female Lewis rats (weighing 120-135 g) were obtained from Janvier Labs and housed at the Experimental Biomedicine facility at the University of Gothenburg, where they were provided with food and water ad libitum. All experiments described were approved by the government animal ethics committee and conducted according to the regulations and guidelines for animal care (EU Directive 2010/63/EU for animal experiments).

### TOL2

TOL2 as defined in EXAMPLE 1 was used in this EXAMPLE 2.

### Induction, Treatment, and Clinical Evaluation of EAMG

For induction of EAMG, rats were anaesthetized with 2% isoflurane supplemented with oxygen. They were injected subcutaneously in both hind footpads and at three sites in the lower back with a total of 80 μg TOL2 or PBS in CFA (Becton, Dickinson and Company) supplemented with 2 mg/ml inactivated Mycobacterium tuberculosis H37RA (Becton, Dickinson and Company), in a final volume of 250 μl.

40 days after EAMG induction, rats were treated with either 10 mg/kg diclofenac (Voltaren, Novartis) administered i.p. or a combination treatment consisting of 10 mg/kg diclofenac administered i.p. followed by an intravenous injection of 250 μg TOL2. Control animals received only PBS.

Body weight was recorded, and the clinical score was observed on a flat bench before and after exercise and graded based on the presence of the following symptoms: tremor, hunched posture, reduced strength/mobility and dropped head. Exercise consisted of 40 s running in a rodent running wheel. EAMG scores were evaluated as follows: 0: normal strength, no symptoms; 1: normal before exercise, symptoms observed after exercise due to fatigue; 2: symptoms present without exercise; 3: severe symptoms at rest, hind limb paralysis, no grip; 4: moribund.

### Results

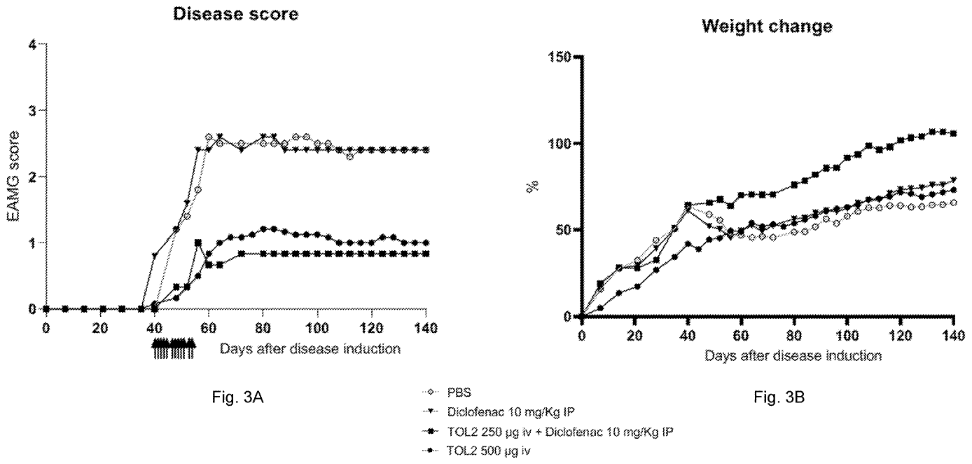

FIG. 3A illustrates the EAMG scores in animals subjected to treatment 40 days after EAMG induction. Intravenous (iv) injection of TOL2 resulted in improvement in EAMG score as compared to controls. Animals receiving intraperitoneal (IP) injection of diclofenac did not show any improvement in EAMG score over PBS-treated control animals. However, the administration of a combination of TOL2 and diclofenac in EAMG rats resulted in an improvement in EAMG scores over PBS-treated controls and also over EAMG rats treated only with TOL2. This was highly surprising since administration of diclofenac alone did not show any improvement over PBS treatment but the combination of TOL2 and diclofenac demonstrated an improvement over only TOL2 administration. Secondly, animals in the reference group received daily doses of 500 μg TOL2 whereas animals subjected to the combination treatment of TOL2 and diclofenac received only 250 μg TOL2. Hence, combining intravenous injection of TOL2 and intraperitoneal injection of diclofenac resulted in a synergistic and dramatic improvement of disease in the treated rats.

The combination of TOL2 and diclofenac also resulted in a larger weight increase in the rats during the 100 days of the trial compared to PBS-treated controls or animals treated with only TOL2 or only diclofenac as shown in FIG. 3B.

### Example 3

In this example the anti-AChR antibody titers in serum of the animals treated in EXAMPLE 1 were investigated.

### Materials and Methods

α-bungarotoxin (Sigma-Aldrich, USA) was labelled with ¹²⁵I using the chloramine T method. Antibodies in test serum samples were quantified using radioimmunoprecipitation assay (RIPA). In brief, for the detection of rat AChR antibodies, rat AChR was prepared from denervated rat muscle and labelled for 1 h at 4° C. with ¹²⁵I-α-bungarotoxin (50,000 counts per minute (cpm)) before incubation with the test serum serial dilutions. The total volume of rat serum used was 2 μl. Then 10 μl of rabbit anti-rat serum were added and incubated overnight at 4° C. Following a centrifugation step, the samples were washed twice with PBS and the remaining radioactivity was measured in a 1470 Wizard γ-counter. The dilutions showing a linear increase were used for the calculation of the antibody titers using the following formula, where specific activity=cpm/fmol labeled antigen:

Antibody titer=(cpm test serum sample−cpm of normal rat serum)/(specific activity of labeled antigen×serum volume in μl×dilution factor)

### Results

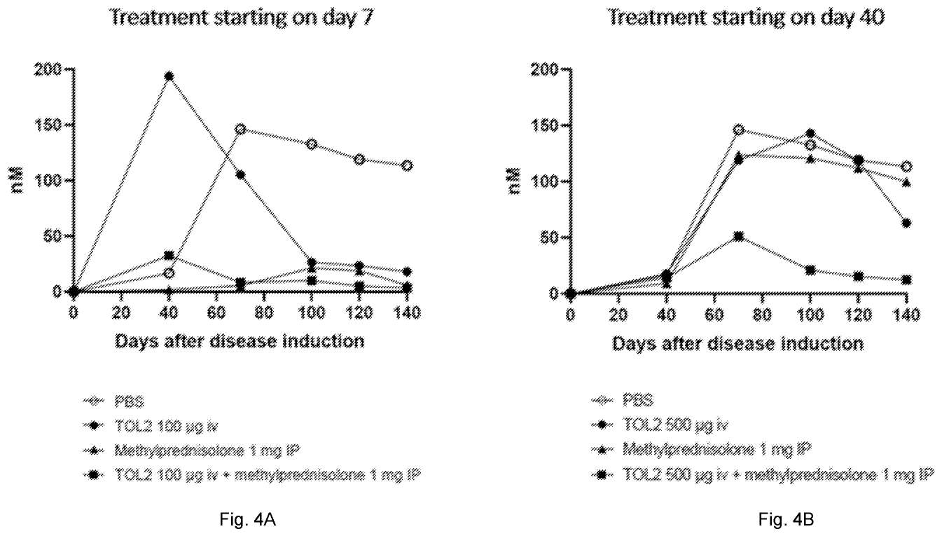

The results are presented in FIG. 4A for rats, in which treatment started on day 7, and in FIG. 4B for rats, in which the treatment started on day 40. As can be seen in the figures, and in particular in FIG. 4B, the combination treatment of TOL2 and methylprednisolone led to a significant reduction in antibody titers as compared to control (PBS) but also as compared to treatment with only TOL2 or only methylprednisolone.

The results in this EXAMPLE 3 thereby confirmed that the positive results seen in EXAMPLE 1 for the combination of TOL2 and methylprednisolone were, at least partly, due to a reduction in antibodies against the pentameric self-protein AChR in the treated rats.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-24. (canceled)

25. A combination comprising an effective amount of a tolerogen and an effective amount of an immunomodulator.

26. The combination according to claim 25, wherein the tolerogen induces, when administered to a subject suffering from an autoimmune disease or having a risk of developing the autoimmune disease, immunological tolerance to a self-protein of the subject that the subject's immune system incorrectly attacks in the autoimmune disease.

27. The combination according to claim 25, wherein the tolerogen induces immunological tolerance to skeletal muscle acetylcholine receptor subunit alpha 1 (nAChRα1).

28. The combination according to claim 27, wherein the tolerogen is selected from the group consisting of SEQ ID NO: 3, 5, 6, 10, and a mixture thereof.

29. The combination according to claim 28, wherein the tolerogen is selected from the group consisting of SEQ ID NO: 5, 6, and a mixture thereof.

30. The combination according to claim 28, wherein the tolerogen is selected from the group consisting of SEQ ID NO: 3, 10, and a mixture thereof.

31. The combination according to claim 25, wherein the immunomodulator is selected from the group consisting of an immunosuppressant, a regulatory T (Treg) cell activator and a combination thereof.

32. The combination according to claim 31, wherein the immunosuppressant is selected from the group consisting of an anti-inflammatory agent, a T cell activation inhibitor, and a combination thereof.

33. The combination according to claim 32, wherein the anti-inflammatory agent is selected from the group consisting of a corticosteroid, a nonsteroidal anti-inflammatory agent, and a combination thereof.

34. The combination according to claim 33, wherein the corticosteroid is a glucocorticoid selected from the group consisting of cortisol, cortisone, prednisone, prednisolone, methylprednisolone, dexamethasone, betamethasone, triamcinolone, deflazacort, fludrocortisone acetate, deoxycorticosterone acetate, aldosterone, beclomethasone, and any combination thereof.

35. The combination according to claim 34, wherein the glucocorticoid is methylprednisolone.

36. The combination according to claim 33, wherein the nonsteroidal anti-inflammatory agent is selected from the group consisting of a nonselective cyclooxygenase (COX) inhibitor, and a selective COX inhibitor.

37. The combination according to claim 36, wherein the nonsteroidal anti-inflammatory agent is a nonselective COX inhibitor selected from the group consisting of ibuprofen, naproxen, diclofenac, mefenamic acid and indomethacin.

38. The combination according to claim 37, wherein the nonselective COX inhibitor is diclofenac.

39. The combination according to claim 36, wherein the selective COX inhibitor is selected from the group consisting of celecoxib, and etoricoxib.

40. The combination according to claim 32, wherein the T cell activation inhibitor is selected from the group consisting of a T-cell immunoglobulin and mucin-domain containing-3 (TIM-3) ligand, a programmed cell death protein 1 (PD-1) ligand, a lymphocyte-activation gene 3 (LAG-3) ligand, and a cluster of differentiation 28 (CD28) antagonist.

41. The combination according to claim 40, wherein the TIM-3 ligand is selected from the group consisting of galectin-9, high mobility group protein B1 (HMGB1), carcinoembryonic antigen cell adhesion molecule 1 (CECAM1), and phosphatidylserine.

42. The combination according to claim 40, wherein the PD-1 ligand is selected from the group consisting of programmed death 1 ligand 1 (PD1L1), PD1L2, a soluble extracellular domain of PD1L1, and a soluble extracellular domain of PD1L2.

43. The combination according to claim 40, wherein the LAG-3 ligand is selected from the group consisting of major histocompatibility complex class II (MHC class II), fibrinogen-like protein 1 (FGL-1), alpha-synuclein, galectin-3, and lymph node sinusoidal endothelial cell C-type lectin (LSECtin).

44. The combination according to claim 40, wherein the CD28 antagonist is selected from the group consisting of cytotoxic T-lymphocyte-associated protein 4 (CTLA4), and Abatacept.

45. The combination according to claim 31, wherein the Treg cell activator is selected from the group consisting of interleukin-2 (IL-2), transforming growth factor β (TGF-β), retinoic acid, rapamycin, butyrate, and any combination thereof.

46. The combination according to claim 45, wherein the Treg cell activator is selected from the group consisting of rapamycin, butyrate, and any combination thereof.

47. The combination according to claim 25, wherein the immunomodulator is selected from the group consisting of a nonselective cyclooxygenase (COX) inhibitor, a glucocorticoid, and any combination thereof.

48. The combination according to claim 47, wherein
the nonselective COX inhibitor is diclofenac; and
the glucocorticoid is methylprednisolone.

49. The combination according to claim 25, wherein
the tolerogen is selected from the group consisting of SEQ ID NO: 3, 5, 6, 10, and a mixture thereof; and
the immunomodulator is selected from the group consisting of a glucocorticoid, a nonselective cyclooxygenase (COX) inhibitor, and combination thereof.

50. The combination according to claim 49, wherein
the tolerogen is selected from the group consisting of SEQ ID NO: 3, 10, and a mixture thereof; and
the immunomodulator is selected from the group consisting of methylprednisolone, diclofenac and a combination thereof.

51. The combination according to claim 25, wherein the tolerogen and the immunomodulator are provided in an administration form for separate administration of the effective amount of tolerogen and the effective amount of the immunomodulator.

52. The combination according to claim 25, wherein the combination comprises a first formulation comprising the effective amount of the tolerogen and a second formulation comprising the effective amount of the immunomodulator.

53. The combination according to claim 25, wherein the tolerogen and the immunomodulator are provided in an administration form for simultaneous administration of the effective amount of tolerogen and the effective amount of the immunomodulator.

54. A method for preventing, inhibiting or treating an autoimmune disease, the method comprises administering an effective amount of a combination according to claim 25 to a subject in need thereof, wherein the tolerogen induces, when administered to a subject suffering from the autoimmune disease or having a risk of developing the autoimmune disease, immunological tolerance to a self-entity of the subject that the subject's immune system incorrectly attacks in the autoimmune disease.

55. The method according to claim 54, wherein the autoimmune disease is myasthenia gravis.


SEQUENCE LISTING (10 sequences)

SEQ ID NO: 1 - protein, 457 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 2 - protein, 235 aa, Homo sapiens
SEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 3 - protein, 210 aa, Homo sapiens
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 4 - protein, 12 aa, Lymnaea stagnalis
DVSGVDTESGAT

SEQ ID NO: 5 - protein, 210 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRL

SEQ ID NO: 6 - protein, 211 aa, synthetic construct
MSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRL

SEQ ID NO: 7 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 8 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 9 - protein, 482 aa, Homo sapiens
MEPWPLLLLFSLCSAGLVLGSEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 10 - protein, 211 aa, synthetic construct
MSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL